(19) United States
(12) Patent Application Publication
(10) Pub. No.: US 2018/0055660 A1
(43) Pub. Date: Mar. 1, 2018

(54) DIAGNOSTIC AND THERAPEUTIC APPROACHES FOR SPINAL DEFORMITIES

(71) Applicants: John MACMAHON, Exeter, NH (US); Luke STIKELEATHER, Oakton, VA (US); Tom SAUL, Moss Beach, CA (US); Jeremy BOYETTE, Campbell, CA (US)

(72) Inventors: John MACMAHON, Exeter, NH (US); Luke STIKELEATHER, Oakton, VA (US); Tom SAUL, Moss Beach, CA (US); Jeremy BOYETTE, Campbell, CA (US)

(21) Appl. No.: 15/792,216

(22) Filed: Oct. 24, 2017

Related U.S. Application Data
(63) Continuation-in-part of application No. PCT/US2016/060658, filed on Nov. 4, 2016.
(60) Provisional application No. 62/250,958, filed on Nov. 4, 2015.
(60) Provisional application No. 62/412,150, filed on Oct. 24, 2016.
(60) Provisional application No. 62/500,961, filed on May 3, 2017.
(60) Provisional application No. 62/562,185, filed on Sep. 22, 2017.

(51) Int. Cl. A61F 2/78; A61B 5/00; A61F 2/68; A61B 5/107

(52) U.S. Cl. CPC A61F 2/7843; A61B 5/4561; A61F 2002/501; A61B 5/1071; A61F 2/68

(57) ABSTRACT

Wearable devices for spinal deformities, including a wearable brace configured to be worn about a patient's torso, at least one inflation bladder carried directly or indirectly by an inner surface of the brace, and positioned such that inflation of the bladder with a fluid applies a pressure to generate a force on the patient at a targeted location, at least one force sensor carried directly or indirectly by a surface of the brace, to measure the force independent of the bladder, and the force sensor adapted to output a signal indicative of a force applied on the sensor.

### CROSS REFERENCE TO RELATED APPLICATIONS

This application is a continuation-in-part application of PCT/US2016/060658, filed Nov. 4, 2016, which claims the benefit of U.S. Provisional App. No. 62/250,958, filed Nov. 4, 2015, both of which are incorporated by reference herein.

This application also claims the priority of the following U.S. Provisional applications, each of which is incorporated by reference herein: U.S. App. No. 62/412,150, filed Oct. 24, 2016; U.S. App. No. 62/500,961, filed May 3, 2017; and U.S. App. No. 62/562,185, filed Sep. 22, 2017.

### INCORPORATION BY REFERENCE

All publications and patent applications mentioned in this specification are herein incorporated by reference to the same extent as if each individual publication or patent application was specifically and individually indicated to be incorporated by reference. This application incorporates by reference herein the disclosure of U.S. Pub. No. 2015/0257915, published Sep. 17, 2015.

### BACKGROUND

Improved diagnostic methods and devices, as well as therapeutic braces, and methods of using them, are needed for individuals with spinal deformities such as scoliosis. A particular need exists to improve the initial assessment of the spinal deformities range of motion and, in particular, what range of motion can be attained in a comfortable and sustainable fashion. A diagnostic tool to provide this information is needed. Improved diagnostics methods and systems are also needed that use a more objective assessment for determining a configuration of a therapeutic brace that is used to treat one or more spinal deformities. Empowering the patient to control their own custom force and/or pressure inputs in a wearable device is also advantageous.

### SUMMARY OF THE DISCLOSURE

The disclosure relates generally to spinal devices, systems and methods, and in some cases to pain diagnosis and/or treatment.

One aspect of the disclosure is a diagnostic method of distorting a patient's spine as part of a brace making procedure, comprising: engaging a torso of a patient (even if covered with clothing) with more than one distender interface, with a first distender interface engaging one side of the patient and a second distender interface engaging a second side of the patient, at least one of the first and second distender interfaces comprising a bladder and a force sensor; sensing pressure in at least one of the first and second bladders; inflating the at least one of first and second bladders; applying corrective forces on the patient with the at least one of first and second bladders; and measuring forces from the at least one of first and second force sensors when the sensed pressure in the at least one of first and second bladders is below an upper pressure threshold.

The method can further comprise imaging the patient after applying the corrective forces to generate a force corrected image, and optionally further comprising creating a model of a therapeutic brace based on the measured forces and force corrected image.

Engaging a torso of a patient with more than one distender interface can comprise engaging the torso with at least three distender interfaces (e.g., five distender interfaces), with at least two placed on the first side of the subject, and at least one placed on the second side.

Measuring forces can comprise measuring forces when the sensed pressure in at least one of the first and second bladders is not above 2.5 psi, and optionally not above 2.0 psi.

One aspect of the disclosure is a diagnostic apparatus for applying corrective forces on a patient's spine while sensing patient information, comprising: a patient apparatus comprising a plurality of distenders extending in a traverse plane, each of the distenders comprising a distender interface, and each of the plurality of distenders supported by a support member, at least one of the distender interfaces comprising a relatively rigid element, a bladder coupled to a pressure sensor, and a force sensor, the bladder and force sensor secured directly or indirectly to an inner surface of the rigid element.

In some embodiments at least one of the relatively rigid elements has a curvilinear configuration. There can be at least three distenders (optionally at least five), with at least two distenders supported by a first support member, and at least one distender supported by a second support member.

The pressure sensor and the force sensor can be in communication with a computer with a computer executable method adapted to relate the sensed force and pressure from each of the distender interfaces.

One aspect of the disclosure is a therapeutic brace for treating a spinal deformity, comprising: a rigid body; and at least one distender interface secured to an inner surface of the rigid body, the at least one distender interface comprising a bladder co-located with a force sensor, the bladder in communication with a pressure sensor (the pressure sensor optionally co-located with the distender interface or spaced from the distender interface), wherein the force sensor and pressure sensor are in communication with a brace module comprising a communication member that is adapted to communicate information indicative of the sensed force and pressure to an external device.

One aspect of the disclosure is a computer executable method stored on a memory, comprising: receiving information indicative of a force sensed by a force sensor on a therapeutic brace for treating a spinal deformity; receiving information indicative of pressure in a bladder on a therapeutic brace for treating a spinal deformity; causing an alert on an external device in communication with the therapeutic brace if the information indicative of the force sensed by the force sensor or the pressure in the bladder drops below a threshold level.

One aspect of the disclosure is any of restoring pads herein, alone or incorporated into any of the therapeutic braces or any of the diagnostic devices herein.

One aspect of the disclosure is a method of obtaining a force or pressure corrected image of a patient, comprising: positioning a patient with a spinal deformity within a standing or sitting frame, the standing or sitting frame comprising one or more distenders, at least one of the or more distenders comprising a bladder; deforming the patient's spine by contacting the at least one bladder with the patient; after the deforming step, measuring at least one of force on the patient and pressure within the at least one bladder; and measuring the deformation of the spine.

Deforming the patient's spine can comprise at least one of moving the one or more distenders toward the patient and inflating the at least one bladder. Deforming can comprise inflating the at least one bladder without moving the one or more distenders. Deforming can comprise moving the one or more distenders with an already inflated bladder into contact with the patient.

Measuring the deformation can comprise imaging the patient after deforming the patient's spine.

Measuring the deformation can comprise measuring displacements of the one or more distenders.

The method can further comprise creating a brace body based on the measured information.

One aspect of the disclosure is a standing or sitting frame for generating a force or pressure corrected image, comprising: a standing frame comprising one or more distenders, at least one of the distenders comprising an inflation bladder disposed in an end region of the distender, the at least one bladder positioned to contact a patient standing or sitting within the frame, and at least one of a force sensor and a pressure sensor adapted to sense force applied to the patient by the bladder and/or pressure within the bladder.

One aspect of the disclosure is a wearable device for spinal deformities, comprising: a wearable brace configured to be worn about a patient's torso; at least one inflation bladder carried directly or indirectly by an inner surface of the brace, and positioned such that inflation of the bladder with a fluid applies a pressure to generate a force on the patient at a targeted location to treat scoliosis; and at least one force sensor carried directly or indirectly by a surface of the brace, to measure the force independent of the bladder, and the force sensor adapted to output a signal indicative of a force applied on the sensor. The force sensor can be carried directly or indirectly by the inner surface of the brace. Resistance of the sensor can change in response to a force applied to the force sensor. The force sensor can be positioned to sense a force applied by the inflation bladder on the patient. The force sensor and the bladder can be co-located between the patient and the brace. The device can further comprise a pressure sensor adapted to sense pressure within the bladder.

One aspect of the disclosure is a wearable device for spinal deformities, comprising: a wearable brace configured to be worn about a patient's torso; at least one inflation bladder carried directly or indirectly by an inner surface of the brace, and positioned such that inflation of the bladder with a fluid applies a pressure to generate a force on the patient at a targeted location; and at least one force sensor carried directly or indirectly by a surface of the brace, to measure the force independent of the bladder, and the force sensor adapted to output a signal indicative of a force applied on the sensor. The force sensor can be carried directly or indirectly by the inner surface of the brace. Resistance of the sensor can change in response to a force applied to the force sensor. The force sensor can be positioned to sense a force applied by the inflation bladder on the patient. The force sensor and the bladder can be co-located between the patient and the brace. The device can further comprise a pressure sensor adapted to sense pressure within the bladder.

One aspect of the disclosure is a system for spinal deformities, comprising: a wearable brace configured to be worn about a person's torso; at least one inflation bladder carried directly or indirectly by an inner surface of the brace, and positioned such that inflation of the bladder with a fluid applies a pressure to generate a force on the patient at a targeted location to treat scoliosis; and at least one force sensor carried directly or indirectly by a surface of the brace, to measure the force independent of the bladder, and the force sensor adapted to output a signal indicative of a force applied on the sensor; and a signal conditioning module with a communication element to communicate force sensor outputs to an external device.

The system can further comprise a pressure sensor adapted to sense pressure within the bladder. The system can communicate pressure sensor outputs from the pressure sensor to the external device. The external device can be adapted to keep a time history of at least one of force and pressure.

The force sensor can be carried directly or indirectly by the inner surface of the brace.

The force sensor can be positioned to sense a force applied by the inflation bladder on the patient.

The signal conditioning module can be housed in a housing coupled directly or indirectly to an outer surface of the brace.

The force sensor and the bladder can be co-located between the patient and the brace.

### BRIEF DESCRIPTION OF THE DRAWINGS

FIG. 1 illustrates an exemplary fixed diagnostic standing frame, which can be used to create a force corrected image of a patient while the patient can provide feedback on the comfort level of being corrected by those forces.

FIGS. 2 and 2A illustrates an exemplary fixed diagnostic sitting frame, which can be used to create a patient specific force corrected image of a patient with comfort feedback.

FIG. 3 illustrates an exemplary wearable brace.

FIG. 4 illustrates an exemplary wearable brace.

FIGS. 5A and 5B illustrate an exemplary wearable brace.

FIG. 6 illustrates an exemplary wearable brace.

FIG. 7 illustrates an exemplary wearable brace with integrated sensor.

FIG. 8 illustrates an exemplary wearable brace with integrated sensor.

FIG. 9 illustrates an exemplary wearable brace with integrated sensor.

FIG. 10 illustrates an exemplary wearable brace with integrated sensor.

FIG. 11 illustrates an exemplary wearable brace with integrated sensor.

FIG. 12 illustrates an exemplary system including a wearable brace and an external device.

FIG. 13 illustrates an exemplary wearable monitoring system in which sensors are integrated into a sensing garment.

FIG. 14 illustrates an exemplary force sensor and/or pressure sensing assembly that can be incorporated into a therapeutic brace (or diagnostic system), optionally for treating at least one scoliosis and pain.

FIG. 15 illustrates a therapeutic brace with an exemplary force and/or pressure sensing assembly secured thereto.

FIG. 16 illustrates a therapeutic brace with an exemplary force and/or pressure sensing assembly secured thereto.

FIGS. 17A, 17B, 17C, 17D and 17E illustrate exemplary screen shots of an exemplary software application that can provide one or more types of information to a user based on sensed information.

FIG. 18 illustrates an exemplary pad, including a pocket with a force sensor disposed therein.

FIG. 19 illustrates an exemplary brace with a plurality of pads secured thereto.

FIG. 20 illustrates an exemplary finger pump that can be used with any pad herein.

FIG. 21 illustrates an exemplary application screen shot on a display of a user device that can show pressure and force readings, and also qualitatively on a spectrum for ease of use.

### DETAILED DESCRIPTION

One aspect of this disclosure relates to diagnostic methods, systems, and devices that can help assist in making a more objective assessment or determination about the initial shape of a wearable therapeutic brace to be used to treat one or more spinal deformities. Every patient can have a different degree of spinal deformity, and thus patient specific braces are desired and more effective. A standard approach for creating a therapeutic brace initially involves either scans or caliper measurements of a patient in a standing or prone position. This provides a gross assessment of the patient's body surface for input into the brace building process. The brace maker then relies on two inputs for determining how much the spine can be corrected while the patient would still be comfortable in that brace. Those two variables are these external geometric measurements and an X-ray of the patient's spine. The orthotist is now drawing only on his training and his experience to produce a brace that can provide a comfortable brace with a satisfactory support or correction of the spine. This is a very subjective step, and relies on the brace maker's experience to create a final brace geometry. As one would suspect, the subjectivity produces a wide range of results. It is accepted in that after building this brace a fitting appointment to trim, pad and adjust the brace requires 2-4 hours. The impact of this subjective process was also noted in the BrAIST Study, a randomized control trial for scoliosis braces published in the NEJM in 2013. In the independent review of BrAIST Bracing Evaluation Committee of 222 braces found that only 68% of braces were satisfactorily customized for each patient. In the same Committee the satisfactory immediate outcome was even lower at 61%.

This disclosure, however, improves upon the standard approach by providing a more objective approach when choosing the therapeutic brace configuration. To help standardize and elevate the quality of scoliosis braces, the methods herein provide simple and accurate means of providing the patient and the orthotist to collaborate and agree on the forces and comfort that are required before taking these measurements. The resulting measurements will then consistently produce comfortable and clinically effective braces for any orthotist. The improved methods herein utilize one or more signals sensed from a diagnostic system that is configured and adapted to interface with the patient. In some embodiments the diagnostic methods include interfacing discrete portions of the patient's torso with separate interface assemblies of a diagnostic system. The interface assemblies apply corrective forces to the patient's torso (similar to how the brace may apply forces to the patient). The brace maker can then use one or more imaging modalities to generate a “corrected scan” of the patient. The brace maker can then use that corrected scan when 3D modeling the therapeutic brace. Rather than relying on subjective years of experience to approximate the forces and geometry that will be necessary to correct the patient's spinal deformity, the systems and methods herein can provide actual force measurements applied to the patient as well as an image of the “corrected” patient. Additionally and provocatively, the inclusion of patient comfort is a big step. It has been shown that comfort drives brace wear time, and that outcomes are driven by brace wear time, therefore comfort is directly related to outcomes. A further benefit is having comfort expectations established up front. Final fitting is 2-4 hour appointments and we expect to reduce the time required to create a brace to treat the deformity and improve the initial fit of the brace.

FIG. 1 illustrates an exemplary diagnostic standing apparatus 100 adapted and configured for use in characterizing the relationships between the magnitude of a distension, the corresponding change in Cobb angle, and the resultant interface pressure associated with the application of such a distension. The apparatus has an exemplary use in an x-ray or other imaging system capable of imaging the position and displacements of the spine as a function of displacement of at least one of the plurality of distenders 101. Apparatus 100 comprises three distenders 101, although more of fewer may be used. The distenders in some embodiments additionally comprise a monitoring member adapted to monitor displacements particularly in the plane including by the two frame elements 102. Each of the distenders 101 interface with a frame structure or element 102. Each distender 101 additionally comprises a distender interface 103 at a distal end of the distender 101, which can include an element that is adapted to conform to a body surface to which it will interface when in use. The distender interfaces may comprise any selection or combination of foams, fluid or air filled bladders, and elements adapted and configured to conform to the contour of the body being evaluated. Each distender interface may additionally comprise any selection or combination of an interface surface providing contrast in the generated image, pressure sensors, and load/force sensors. Pressure and load/force sensors allow for the characterization of the pressures associated with the distenders (e.g., air pressure within a bladder) and forces generated by the distenders on the patient's body. The distender interfaces, even if they do not specifically include a pressure sensor, may be in fluid communication with a pressure sensors adapted to sense pressure.

The system is also adapted to measure or monitor the displacement, location, and/or orientation of the distender and/or the distender interface(s), in a reference 3D space. The distenders interface the stand elements such that they can move towards and away from the patient. The interfaces can be adapted to move with respect to the distender, such as with one or more degrees of freedom. This freedom of movement allows the distender interface to orient itself when the interface it placed in contact with the patient such that the distender interface has a better fit with the patient. Knowing the orientation of the interface surface in a reference 3D space when applying the corrective forces allows the system and/or brace maker to more reliably form the brace so that that portion of the brace has a similar orientation in the final brace. The distender interfaces can have one or more radiopaque markers that allow their location and/or orientation to be monitored when visualized with a radiographic imaging modality. The markers can allow the interfaces to be placed in the reference 3D space. The distender interfaces and distenders can also include additional displacement, location and/or orientation sensing mechanisms or means to be able to track displacement, location and/or orientation of the distender interfaces in a reference 3D space.

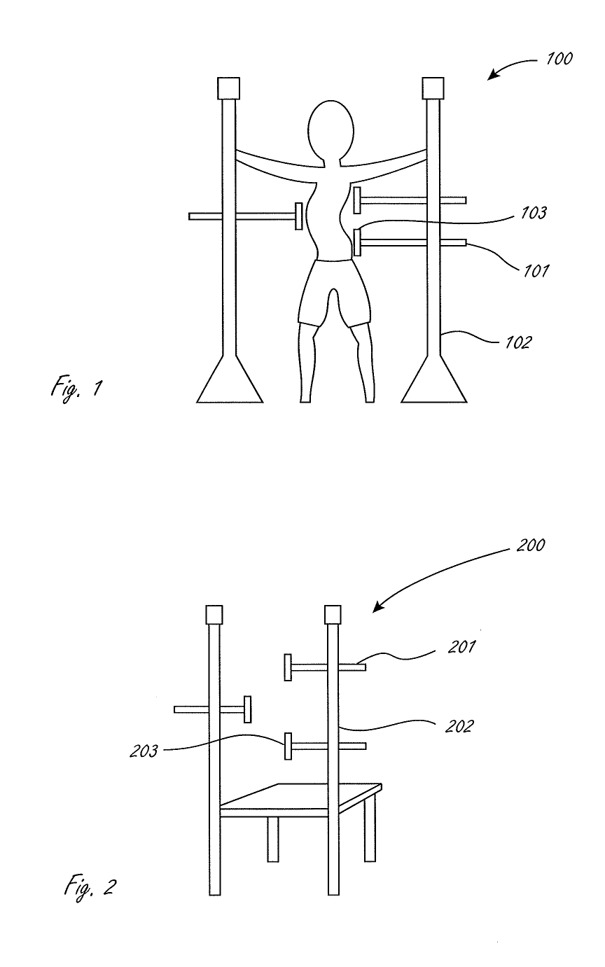

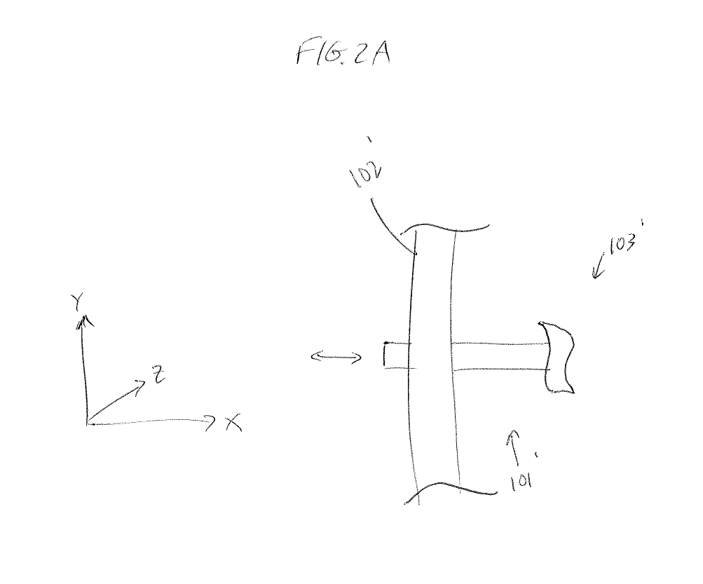

FIG. 2A illustrates that distender/arm 101′ and frame element 102′ can be adapted and configured so that distender 101′ can move in as many as 6 degrees of freedom relative to frame element 102.′ Three axes (X, Y, and Z) of a reference 3D space are shown. Only one type of movement of the distender 101′ is shown by the arrows, but it is understood that the distender 101′ and frame element 102″ can be adapted so that distender 101′ can have more degrees of freedom (e.g, rotation about X axis, movement in X-Z plane, movement in X-Y plane, etc.). Similarly (while not shown in FIG. 2A), interface 103′ and distender 101′ can be configured so that interface 103′ can have as many as 6 degrees of freedom relative to distender 101′. By allowing movement of interface 103′, a better fit can occur between interface 103′ and the patient, which allows for more accurate force and pressure readings taken from the interface 103′. The system can be adapted so that there can be any combination of degrees of freedom between frame element 102′ and distender 101′, and distender 101′ and interface 103′. This allows for degrees of freedom where frame element 102′ interfaces distender 101′, as well as where distender 101′ interfaces with patient interface 103′.

In an exemplary procedure using the exemplary apparatus of FIG. 1, a patient is positioned, such as by standing, between the frame elements 102. The distender elements 101 and interfaces can then be adjusted (such as by utilizing any of the degrees of freedom set forth above) so that the distender interfaces 103 interface with discrete portions of the torso as illustrated such that no or minimal pressure is applied by the distender elements 103. An image can then be acquired using an imaging device, such as an x-ray machine, to generate an uncorrected image of the spine. Distender elements 101 can then be individually adjusted such that “corrective” forces from the interface 103 are applied at discrete locations on the torso, and optionally with an upper pressure limit, such as 2.5 psi or 2 psi, which can be sensed using any of the pressure sensors herein. One or more images of the patient can then be acquired as a “force corrected” image of the patient. The upper pressure limit, such as 2.5 psi or 2 psi limitation will minimize the likelihood of the patient developing ulcers. The bladders are inflated to maximize patient comfort in the effort to evenly distribute the correcting force. The upper pressure limit can be utilized during the diagnostic process such that forces applied by the therapeutic brace will not be greater than the upper pressure limit. In some embodiments force sensors associated with each of the distenders 103 measures force at the location of the interface. These force readings, along with the corrected image of the patient, can then more objectively be used by the brace maker to arrive at a configuration of a therapeutic brace that will as closely as possible create the “corrected image” of the patient when worn by the patient. The apparatuses and methods herein, by sensing force and/or pressure, allow the brace maker (or software if a portion is automated) to know the forces that can (which will during the therapy be applied by the therapeutic brace) adjust the patient's torso into a desired configuration to optimize fit and function, as a brace too tight would not be worn. Feedback during fitting in the apparatus provides greater confidence of the fit, form and clinical benefit, while reducing a subjective step. The order of the method steps can be modified in some procedures, and not every step necessarily needs to be carried out in the procedures.

In this “corrected image” model, it can be externally scanned as well as incorporating imbedded sensors similar to Hall sensor probes or RF proximity sensors, like Polhemus motion tracking, to know the relative position of the shapes engaged against the patient. This “corrected image” may also be attained by embedded or external cameras and applying the principles of photogrammetry. Photogrammetry involves algorithms that stitch multiple images into a 3D image.

One feedback that connects the patient to the brace benefit is the fact that they will be getting immediately taller with a properly fitted brace. The stand can also actively measure the patient's height while the patient is put into a “Corrected Posture.” These measurements may include but are not exclusive to passive suspended measures from above or active in the form for example ultrasound or proximity sensors to the patients head.

In alternate procedures, more images may be acquired at more than two distensions and relationships generated comprising the relationship between measured Cobb angle, distensions, and interface pressures and forces. For the purposes of such diagnostic activities more than an upper pressure limit, such as 2 psi, may be applied for short periods of time. Such measurements are useful in determining a contour for a therapeutic brace, which limits pressures to less than 2 psi for some portion of time while applying a maximum distension during normal activities. Such measurements additionally provide information on the effectivity of a given distension as time goes on when using a therapeutic brace as described herein.

When the conformal element 103 comprises an element fabricated and configured to conform to the contour of the body being evaluated, an exemplary way of fabricating the element is via 3D printing.

In some embodiments one or more distender interfaces 103 may be hydraulic.

The “Force Corrected” scans (imaging) described herein result in an objectively acquired 3D imaged model, or in some cases a 2D X-ray, which will allow a beginner or less experienced brace maker to get experienced brace maker models. Integrating an imaging system into the design process would further accelerate and streamline the results and adoption of the system. Known imaging modality systems can be used.

Exemplary benefits of recording and correcting with both force and pressure are that forces correct the geometry, while pressure on the patient determines the ergonomic comfort. Combining these two is particularly powerful to get the most geometric benefit with ergonomics that would provide comfort. Brace fit is very relevant since patients may be prescribed to wear the brace for up to 20 hours.

The diagnostic apparatuses herein can apply forces to correct a spinal deformity while allowing subsequent imaging to produce “Force Corrected” scan to be performed, imaging may be 2D or 3D.

The diagnostic apparatuses herein can apply pressures to correct a spinal deformity while allowing subsequent imaging to produce “Pressured Corrected” scan to be performed, imaging may be 2D or 3D.

The diagnostic apparatuses herein can apply forces and pressures to correct a spinal deformity while allowing subsequent imaging to produce “Force and Pressure Corrected” scan to be performed, imaging may be 2D or 3D.

The diagnostic apparatuses herein can include geometric bounds to support the force and/or pressure correcting members as well as in integrated scanning system to produce the subsequent imaging.

The diagnostic apparatuses herein can include geometric bounds to support the force and pressure correcting members may be in communication with a remote sensors and controller to allow imaging to be performed either remotely by a center of excellence to a remote center or to be performed in an x-ray venue.

Any of the distenders interfaces herein can include any combination of bladders and sensors herein (e.g. pressure or force sensors), to provide any of the feedback information described herein.

FIG. 2 illustrates an alternative to the standing apparatus of FIG. 1, and configured to allow the patient to sit during imaging. Sitting apparatus 200 can be the same as the standing apparatus of FIG. 1 in all other regards. The sitting apparatus 200 comprises a fixed diagnostic sitting frame 202. Interfaced with the sitting frame 202 are a plurality of distenders 201, as illustrated three distenders are used. In other alternatives more or less distenders may be used. Each distender may comprise any combination of displacement sensor, pressure sensor, and load/force sensor. In some embodiments some or all distenders may comprise no sensing elements. Each distender element 201 comprises a distender interface 203 which may additionally comprise one or more pressure sensors and/or one or more load sensors. In an alternate to the embodiment illustrated, the two distender interfaces 203 on the right may be attached to a single distender unit. In some embodiments the distenders 201 may be adjusted by hand, in others they may be comprised of electrically or hydraulically driven linear activators.

Any of the distenders and distender interfaces described herein may be used in any of the systems herein, such as apparatuses 100 or 200.

In an alternate embodiment to that of FIG. 1 or FIG. 2, the apparatuses may be configured for use on a reclining patient. In such an embodiment the components comprising the system may additionally be fabricated for use in an MRI.

FIG. 3 illustrates a portable and wearable diagnostic brace 300 configured and adapted for use in an imaging system. The portable diagnostic brace 300 comprises a brace structure 302. The brace structure 302 is fabricated of a stiff material capable of supporting the loads generated by the plurality of distenders 301, which can be like any of the distenders herein. As illustrated, system 300 comprises eight distenders 301, but other numbers may be used as described herein with fixed systems, such as at least one distender. Each of the distenders 301 may comprise no or any combinations of displacement sensor, pressure sensor, and load/force sensor, any of which may be co-located with any other. Each of the distenders 301 comprises a distender interface 303 which in turn may comprise features as described herein for other distender interfaces described herein. The brace 302 additionally comprises an optional window 304 to allow for x-ray or other imaging modality lucency. The portable diagnostic brace 300 in addition may comprise an optional RF interface 305 configured to transmit information from the sensors comprised in the system to a remote recording and/or processing device not shown.

As illustrated, the distenders are disposed on the inside of the brace, but in alternate embodiments they may be disposed on the outside of the brace, or in some embodiments may be disposed partially inside and partially outside the brace.

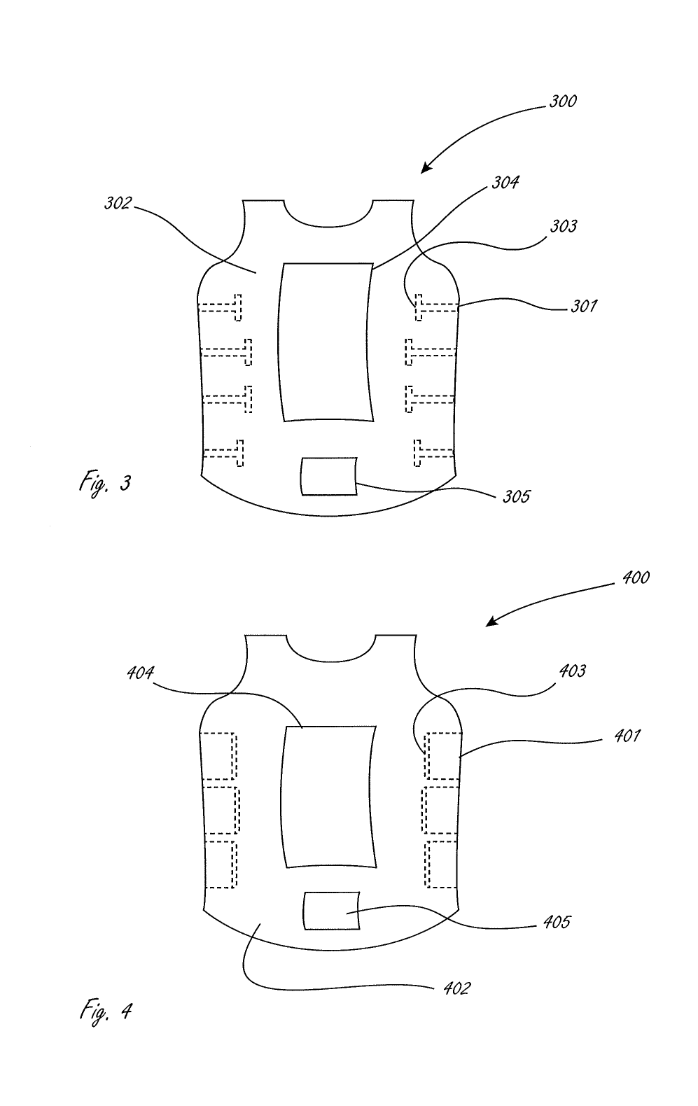

FIG. 4 illustrates portable diagnostic brace 400 similar to that of system 300. In the system of FIG. 4 the at least one distender 401 (six shown in this example) include at least one hydraulic or pneumatic bladders, and the distender interfaces 403 are the interface surface of the bladder, all included on brace structure 402. The at least one distender is in this embodiment directly or indirectly carried by an inner surface of the brace body 402. Each distender 401 may comprise a pressure sensor and/or a displacement sensor for monitoring the pressure on the distender interface 403 and/or changes in position of the distender interface respectively. The one or more distenders 401 may also, or alternatively to the pressure sensors, include one or more force sensors. A force sensor can be co-located with a bladder and between the patient and the brace body, wherein the sensor is adapted to sense force applied to the patient by the bladder. In some embodiments an ultrasonic transducer is disposed in the bladder, or otherwise associated with the bladder, to allow for the monitoring of displacement and/or position of the distender interface surface 403. In some embodiments bladders are inflated manually, such as by a syringe or hand or powered pump. In others, the inflations may be controlled by a manifold comprising controllable valves and sourced by a pump. Inflation systems have not been illustrated. The brace 400 also comprises an imaging system lucent window 404 and an optional RF interface 405 capable of transmitting information from the sensors comprised in the system to a remote recording and/or processing device also not shown.

In alternate embodiments to those of FIGS. 3 and 4, the window 404 may be filled or replaced by an ultrasound sensing system capable of monitoring the conformation of the spine sufficiently to calculate a Cobb angle.

The diagnostic braces set forth herein can also be used as therapeutic braces in the treatment of scoliosis. For example, braces in FIGS. 3 and 4 can be used as therapeutic braces as well, wherein force is applied by the one or more bladders at targeted locations on the patient in the treatment of scoliosis. The sensors can provide feedback during the treatment.

An exemplary advantage of the portable systems is the capability to monitor the variation and magnitude of the interface pressure as a function of activities of daily living. Such measurements may be used to define an optimal shape for a therapeutic brace for daily use.

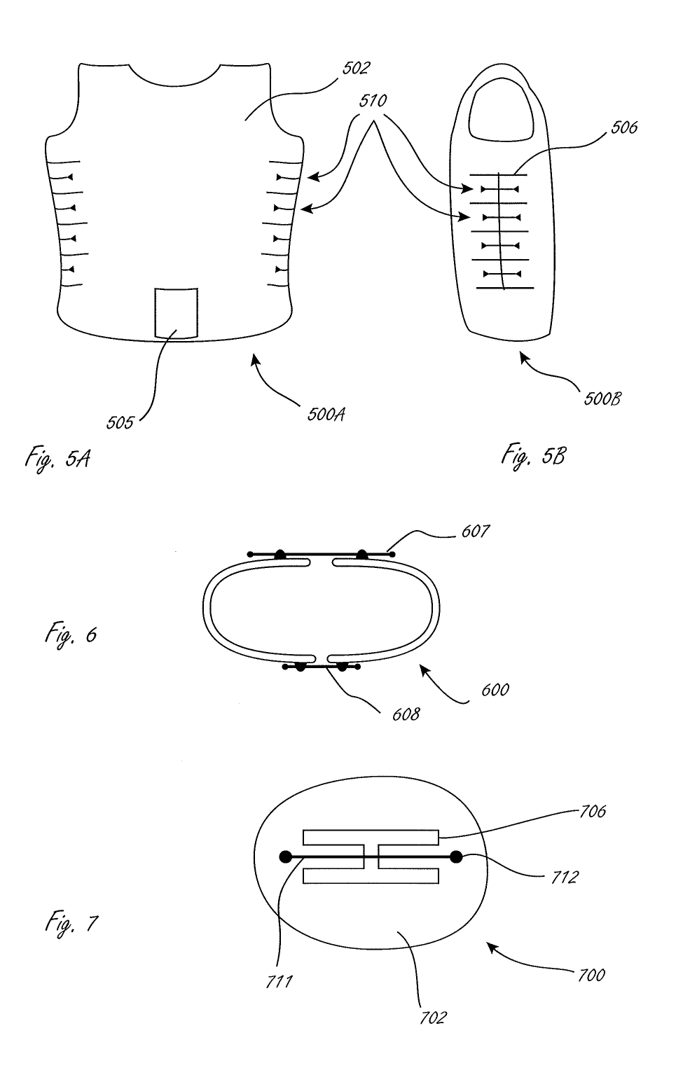

FIGS. 5A and 5B illustrate a therapeutic brace system 500 for daily use. The brace system 500 comprises at least one integral pressure sensor carried directly or indirectly by the brace body 502 (body may also be referred to herein as structure) for monitoring an interface pressure, which should be less than 2 psi on average for reasons as indicated herein. The integral pressure sensors 510 of brace 500 comprise sections of a slit 506 in the brace structure 502 spanned by a strain element or tension monitor 510. In some embodiments the strain element 510 is adjustable allowing for adjustment of a confirmation of the surface of the brace structure 502. The brace 500 comprises a signal conditioning module 505 with optional RF communications. In some embodiments the optional RF module is configured for communications to a cell phone to allow monitoring of interface pressures.

FIG. 6 illustrates another embodiment of a therapeutic brace 600. The brace 600 comprises tension monitors either in the closure 607 or closures and or in a seam opposite the closure 608. When the tension monitor is incorporated on the seam, multiple sensors may be incorporated. In an alternate embodiment the seam may run along the edges of the brace 600. FIGS. 7 thru 12 illustrate a number of possible embodiments for pressure sensors capable of integration into the structure of a therapeutic brace. Such structures should require minimal or no additional thickness in the brace structure.

FIG. 7 illustrates a therapeutic brace integral pressure sensor 700 similar to that illustrated in FIG. 5. Integral pressure sensor 700 comprises a slit 706 in the brace structure 702. A portion of the slit is spanned by a strain wire 711, which is affixed to two strain wire anchors 712 integrated into the brace structure.

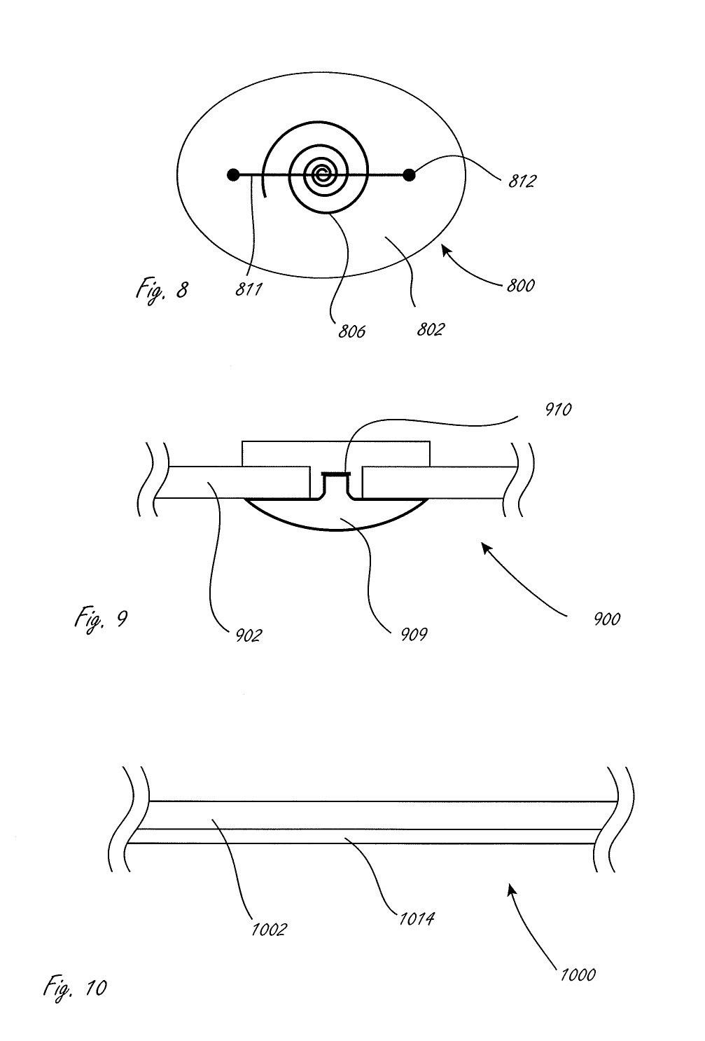

FIG. 8 illustrates an alternate therapeutic brace integral pressure sensor 800. In sensor 800 the slit 806 in brace structure 802 has a spiral shape. The strain wire 811 spanning the slit 806 and associated anchors 812 are similar to those shown in FIG. 7.

FIG. 9 illustrates another alternate therapeutic brace integral pressure sensor 900 integrated into the brace structure 902. The structure comprises a pressure gauge or force gauge 910 integrated with a fluid or solid interface 909 respectively.

FIG. 10 illustrates yet another embodiment of a therapeutic brace integral pressure sensor 1000. In the sensor 1000 the inner surface or portions of the surface of the brace 1002 are covered with a pressure film 1014.

FIG. 11 illustrates yet another embodiment of a therapeutic brace integral pressure sensor 1100 comprising sensor 1113 embedded in a slit in the brace structure 1102. In such embodiments the sensing material of sensor 1113 may comprise a conductive elastomeric material whose resistance across a given path will change as a result of deformations associated with surface pressure on the brace local to the sensor. Alternatively, the material may be a dielectric with conductive surfaces, and the sensor used in a capacitive mode.

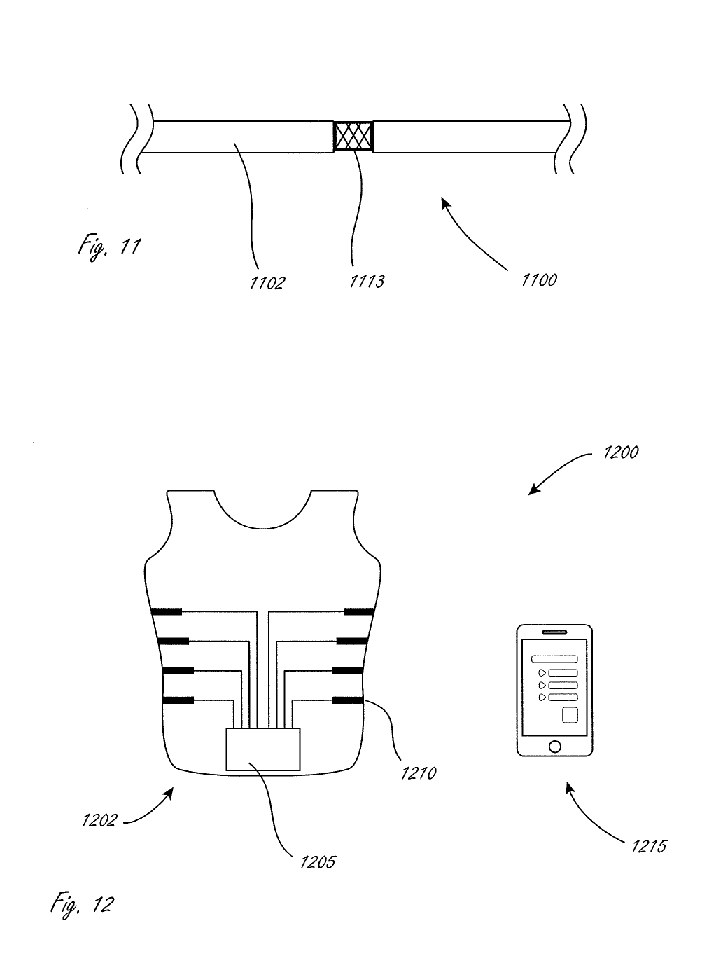

FIG. 12 illustrates a therapeutic brace system 1200 comprising any or any combination of the sensors described above. The system comprises brace 1202, which comprises a number of integrated pressure sensors 1210 carried directly or indirectly by a surface of the brace body. The pressure sensors 1210 are in communications with a signal conditioning module with an optional RF communications feature 1205 on the brace structure and a brace monitoring device 1215. As illustrated the brace monitoring device can be a smartphone or comparable device running an application to periodically record the sensor outputs.

Brace 1202 can be modified to include one or more bladders like in FIG. 4, which can be carried directly or indirectly by the inner surface of the brace body. Brace 1202 can include one or more force sensors (which may replace the pressure sensors), each of which can be co-located with an inflatable bladder, the force sensor adapted to generate an output that is indicative of the force applied to the patient by the bladder.

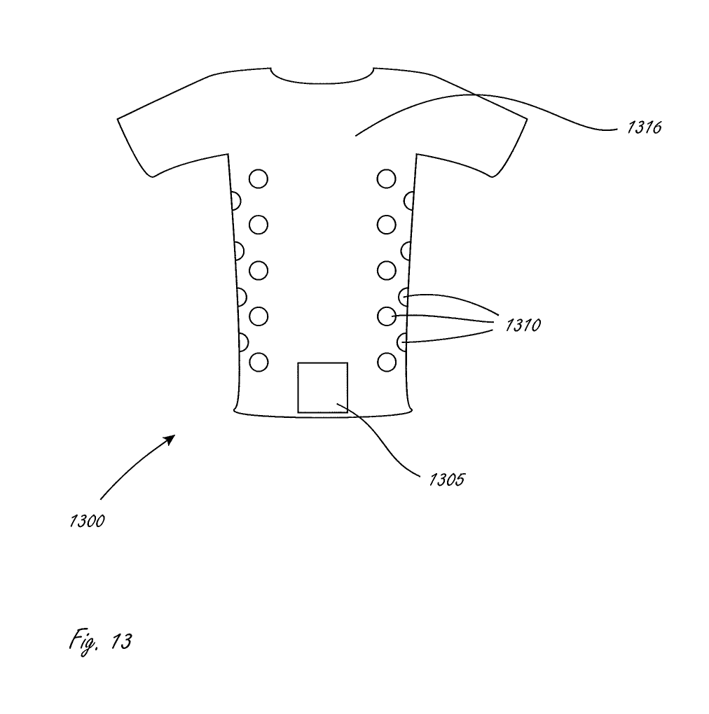

FIG. 13 illustrates an embodiment for a brace pressure monitoring system in which the pressure sensors are integrated into a pressure sensing garment 1300. The garment comprises a base garment 1316 comprising a set of pressure sensitive points 1310. Such a garment may comprise a material which provides some contrast in an imaging modality to allow for assessment of where the garment is in the image. The garment comprises a signal conditioning module with optional RF communications 1305.

Any of the braces herein that include one or more pressure sensors can also include one or more force sensors, which can be co-located with an inflation bladder to be able to sense force applied by the bladder to the patient. The inflation bladder and force sensor can be carried directly or indirectly by an inner surface of the brace body. Braces herein are configured to be worn about a patient's torso.

FIGS. 1 and 2 illustrated (and the text above described) exemplary diagnostic apparatuses. It is noted that for any of the diagnostic apparatus herein each of the distender interfaces can have individually controlled pressures and or forces, or they can all be the same.

In some embodiments the diagnostic apparatus is adapted such that at least three distenders can be used, with at least two on one side and a third on the other side. In some embodiments the diagnostic apparatus includes at least five distenders, each with its own distender interface, with three on one side and two on the other side. In some embodiments the apparatus is adapted such that the five distenders can each be moved up and down on the apparatus to allow for the distender interfaces to be positioned at a variety of desired locations on the patient. And as patient height varies, being able to move them up and down provides an ability to engage the distenders with the patient at whatever the desired anatomical location. In some embodiments those locations are on in the cervical and thoracic regions of the patient.

In some advantageous embodiments, the distender interfaces each include a relatively rigid member, and an inflatable bladder radially inside and secured to the rigid member. The apparatus can be adapted such that the bladder can be filled with a fluid (e.g., liquid or gas) or a gel or gel-like material. The distender interfaces shown in FIGS. 1 and 2 are illustrated as elongate in the superior/inferior direction, with rectangular shapes. The disclosure herein includes embodiments in which the relatively rigid member for any of the distender interfaces can have a curvilinear configuration, an example of which is shown as interface 103′ in FIG. 2A. The relatively rigid members can have configurations that will better correspond to the portion of the body to which they are being applied, which can result in more comfortable fittings and more accurate force readings from force sensors. The configurations of the relatively rigid members can vary, and can be a variety of shapes, such as rectangular and triangular. In some embodiments one or more of the distender interface relatively rigid members are curvilinear sections that can be similar in configuration to sections or material that have been cut out of a therapeutic brace, and are intended to mimic sections of a brace that might be adjacent the portion of the body to which the forces are applied. For example, starting with a therapeutic brace, discrete sections can be cut from the brace at locations where the distender interfaces are intended to engage with the patient. The cut brace sections can be the relatively rigid members. Similarly, if a known curvilinear configuration is desired for one or more of the plurality of interfaces, a template shape/configuration can be established and simply used repeatedly as one of the plurality of rigid members. Theoretically the apparatus could have a plurality of distender interfaces, each of which has a different configuration. Alternatively, the configuration of one or more rigid members can be designed to be patient specific, and can be obtained from, for example without limitation, an image of the patient, or an existing brace.

The relatively rigid members can of course by manufactured using a variety of manufacturing techniques, and may be made from many different materials.

In some embodiments there can be different sets of sizes for the distender interface rigid members. Patients vary by size and it can be advantageous to have different sizes for the interfaces to choose from during the fitting process. For example without limitation, for any or all of the distender interfaces, there can be small, medium, and large sizes, each of which can be scaled accordingly.

The disclosure herein thus includes distender interface rigid members that can have configurations that can each be different from at least one other distender interface rigid member configuration, as well as different sizes for each of the individual rigid members.

Any of the rigid members can be sold or packaged in a kit of other rigid members, which may have the same or different configurations and/or sizes.

It is worth repeating that any or all of the distender interfaces can be associated with, in addition to relatively rigid members, an inflatable bladder in communication with a pressure sensor, and a force sensor. Any of the bladder/force sensor assemblies herein can be used in any of the diagnostic apparatus herein.

In any of the embodiments herein, one or more of the distender interfaces can have radiopaque material therein, allowing the material to be easily identified when images using certain imaging modalities (e.g., X-ray).

The concepts and principles described with respect to the standing or sitting diagnostic apparatus herein (e.g., FIGS. 1 and 2) can be applied to other applications. For example, the diagnostic descriptions here can be applied to pre-operative surgical assessments for any number of conditions, such as kyphosis. For example, a surgeon can use a diagnostic apparatus, or an appropriately modified diagnostic apparatus, on a patient pre-op under fluoro on an operating table to measure forces and stiffness of the spine analytically. In these alternative applications the diagnostic apparatuses can be used in the same or similar ways, such as inflating one or more bladders and measuring the forces.

Once the diagnostic process has taken place, and a brace maker creates a model for the therapeutic brace based on the process above (e.g. using force measurements and/or the force corrected image), the rigid material for the therapeutic brace can be created. One of the aspects of the disclosure herein is therapeutic braces that allow for the brace to apply the necessary forces to the patient even after the patient's body may change. After a period of time of wearing a therapeutic brace (which applies forces on the spine), the patient's spine will begin to realign. Over time, due to the spine realignment, the brace may not be applying the desired forces on the patient for continued realignment. Braces herein are adapted to be able to repeatedly modified such that adequate forces are applied to the patient, and this can be done without reshaping the general configuration of the overall brace (although brace reshaping may in fact occur).

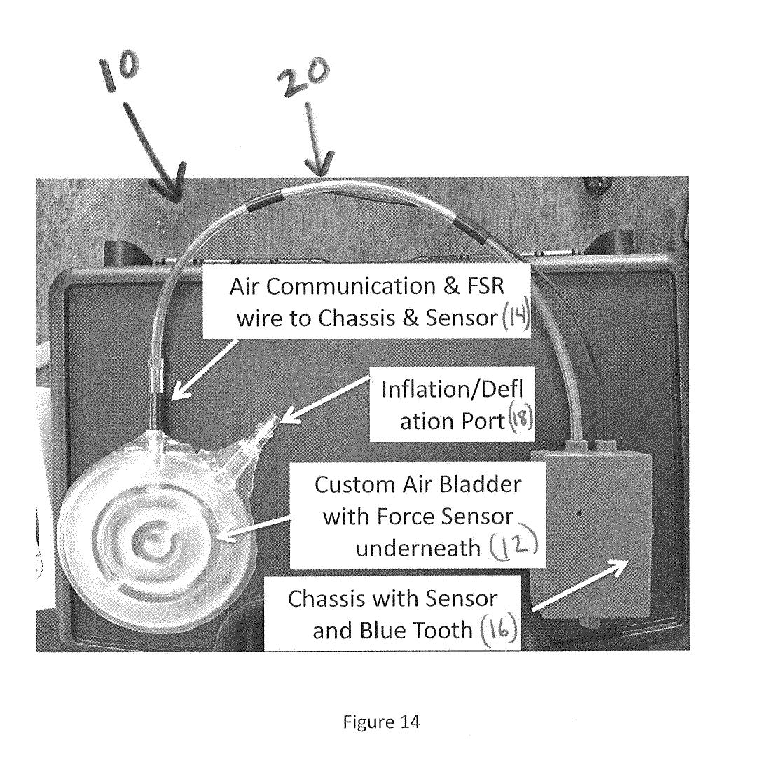

One manner of accomplishing this is described generally above with the therapeutic brace. FIGS. 14-21 illustrate exemplary therapeutic braces that can be modified over time to accommodate spinal realignment and desired forces over time on the patient's spine. FIGS. 14-21 describe exemplary braces that include at least one distender interface, the at least one distender interface including at least one inflatable bladder associated with a pressure sensor, and a force sensor associated with the inflatable bladder. The distender interface(s) are generally secured to the brace at locations where the brace marker wants to apply corrective forces to the patient's spine, and there can be one or more distender interfaces incorporated into the brace. As shown in embodiments in FIGS. 14-21, the interfaces include an air bladder and a co-located force sensor, although in alternative embodiment it is conceivable not to have a force sensor. The bladder is in communication with a pressure sensor, which can be incorporated into the distender interface, or which can be spaced apart from the distender interface. In the embodiments in FIGS. 14-21, the distender interfaces are secured to an inner surface of the brace body. In some embodiments the distender interfaces can be adapted to be releasably secured to the brace body (at least temporarily), such as with an adhesive material that is exposed upon removal of a cover material.

An exemplary advantage to have the ability to measure both pressure and force is that initially, the brace can be put on the patient without any inflation (zero pressure) and force applied at the location of the force sensor (i.e., the bladder) can be sensed. Although it is conceivable that systems can be developed and used that include only a pressure sensor without a force sensor, even if they do not provide all of the functionality of other embodiments herein. Alternatively, some embodiments may be able to rely only on force measurements, and may not include a pressure sensor.

FIGS. 14-21 illustrate exemplary distender configurations, including exemplary distender interface configurations.

Over time, the patient's spine will slowly realign due to the corrective forces being applies, and measured forces and bladder pressure will decrease. To continue to adjust the spinal curvature, the bladders can be inflated with additional fluid to achieve the desired pressure and/or force. Additional fluid can be delivered into the bladders until an upper pressure limit is reached (e.g., 2.5 psi, 2 psi) or until a desired force is a measured. Once the bladder(s) are filled to a desired level, the brace can apply corrective forces until an additional refill is needed or desired.

If more than one bladder is incorporated into the brace, the bladders can have different sizes and/or configurations as desired.

As illustrated in FIGS. 14-21, the systems can be adapted to communicate sensed force and/or pressure with an external device, such as, for example, a handheld device (e.g., smartphone). The handheld device and the brace can be adapted such that sensed information is communicated to the external device, and software (computer executable methods) on the external device can monitor, store, track, and/or display the information or information derived from the communicated information.

The software can be adapted to monitor, store, track, and/or display the sensed pressure or information indicative of sensed pressure.

The software can be adapted to monitor, store, track, and/or display at least one aspect of the patient's breathing cycle, or information indicative of the breathing cycle.

The software can be adapted to monitor, store, track, and/or display the sensed force or information indicative of sensed force.

The software can be adapted to monitor, store, track, and/or display the body position using one or more accelerometers.

This disclosure also includes computer executable methods (e.g., algorithms) that can be stored on remote devices (e.g., smartphones) and provide information to a patient based on information sensed by one or more sensors on a therapeutic (or diagnostic) brace. For example, an App can be downloaded to a smartphone, and the app can visually display and/or store at least one of pressure and force, or information that is indicative of pressure and force. The computer executable code can also be adapted to provide notifications and alerts to the user (e.g., pressure is too low, force is too low, etc.), and can also keep track of compliance.

Software can track force and/or pressure and initiate an alert to the user if one or both are below a level that suggests a bladder refill is desired. In this way the user need not attempt to figure out when to add fluid, as the sensed information provides for a more accurate assessment. The external device can have a processing component adapted to control the execution of one or more computer executable methods (also referred to herein as software or algorithms) stored in memory on the external device.

In any suitable embodiment, a bladder can be adapted to be filled with a hand pump or finger pump, for example.

In any suitable embodiment, the distender can be secured to the bladder in a number of ways. For example without limitation, the force sensor can be disposed on a surface of the bladder, within a pocket of the distender, or a force sensor can be printed on the portion of the bladder that gets inflated.

FIG. 14 illustrates an exemplary sensing and dynamic correction assembly, which can be integrated into any type of therapeutic brace. Assembly 10 includes an interface portion 12 that includes a fluid bladder co-located with a force sensor. Interface portion 10 includes port 18 that is adapted to be in communication with a fluid source (e.g., an air source) to inflate the bladder with fluid. Assembly 10 also includes a fluid line and electrical connection 14 that are coupled to interface portion. The fluid line is in fluid communication with the bladder, and a pressure sensor within member 16. The pressure within bladder can thus be sensed, and in this embodiment with a pressure sensor spaced from the bladder and not co-located with the bladder. The electrical connection is electrically coupled to the force sensor in portion 12.

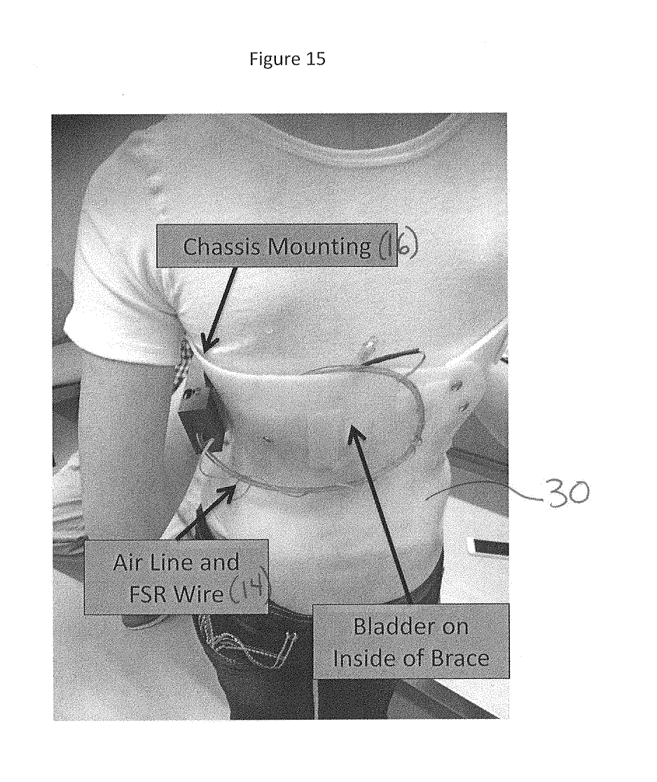

FIG. 15 illustrates an exemplary manner in which assembly 10 shown in FIG. 14 can be coupled to therapeutic brace 30. In this merely exemplary embodiment, chassis 16 is mounted to an external surface of brace 30, at a location under the arm. The interface portion 12 (which includes the bladder and force sensor), and which is hidden in FIG. 15, is coupled (e.g., temporarily) to an inner surface of the brace and at a desired location to apply corrective forces to the patient.

An exemplary method of using assembly 10 and the brace is now described. The first stage is the fitting of the initially created brace. The force sensor can be placed alone on the inside of the brace to confirm the fit of the brace is sufficient to develop the forces at the desired anatomical locations (peak of scoliosis/kyphosis curve). The brace can then be modified or adjusted using known techniques based on this initial feedback from the force sensor. In a second step, the force sensor and bladder assembly is attached to the brace. The brace is then put on the patient's body and force is confirmed with an empty fluid bladder. Once the desired fit is achieved, the fluid bladder is inflated to concentrate and optimize support of the brace on the target anatomical locations.

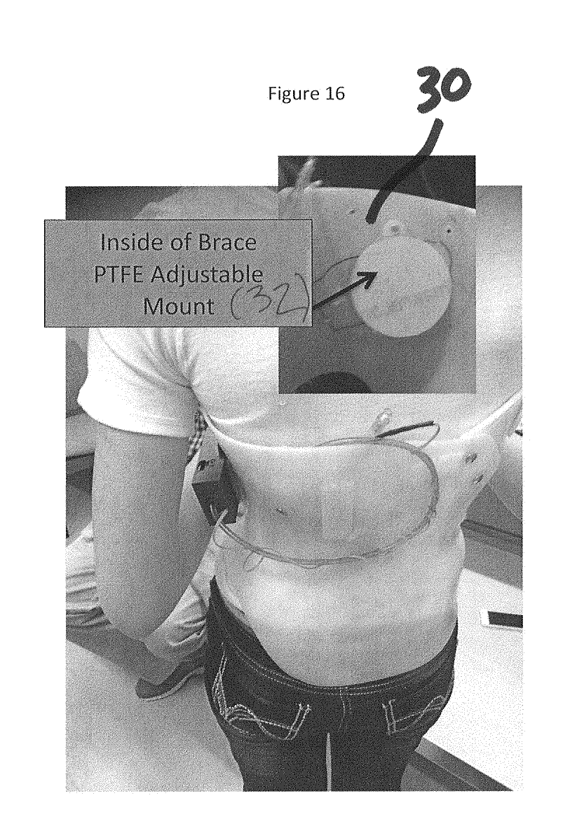

FIG. 16 illustrates an exemplary view of an inside of the brace, showing an adjustable mount 32. The interface portion 12 can be secured to mount 32, which can provide comfortable skin contact.

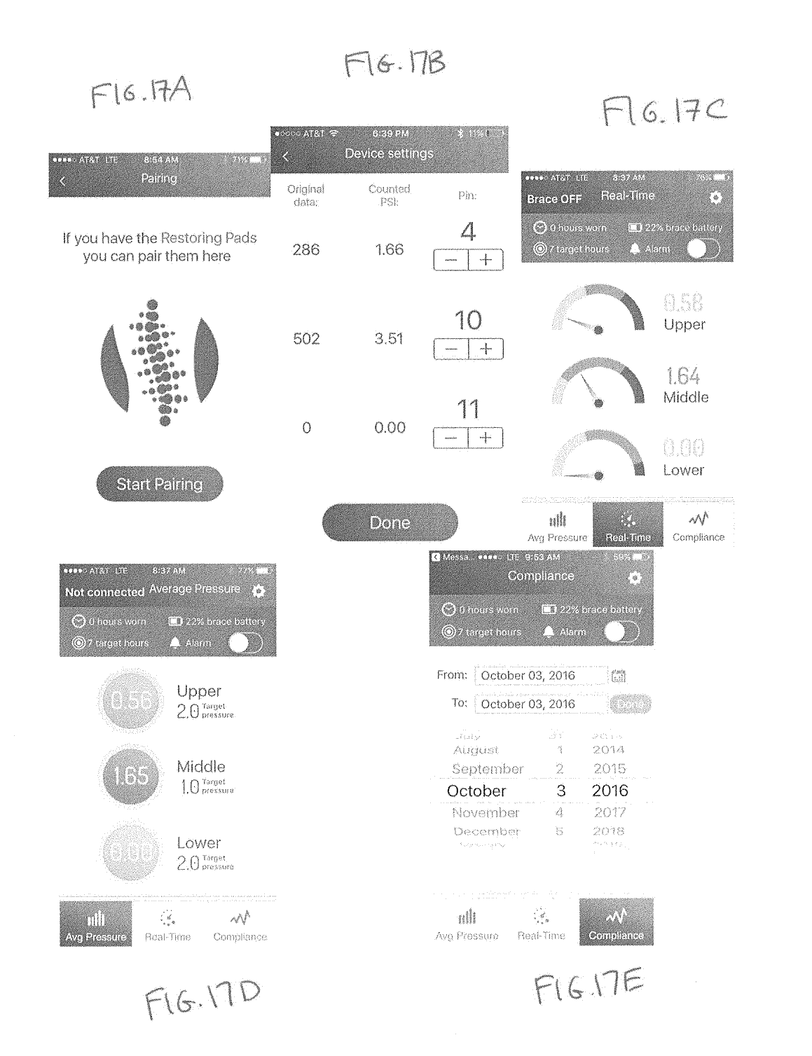

FIGS. 17A-17E illustrate screen shots of exemplary computer executable methods that are adapted to visually present a variety of information to a user, which in some figures are based on information sensed from a force and/or pressure sensor. FIG. 17A illustrates a pairing screen shot where the external device is pairing with a sensing assembly coupled to a brace. FIG. 17B illustrates a calibration screen that shows a pressure reading based on sensed pressure. FIG. 17C illustrates real-time sensing at three locations with real time pressures shown in PSI. The display can alternatively show force readings. FIG. 17C also shows a qualitative visual element that relates the sensed parameter to a spectrum, color coded to indicate different levels of readings. FIG. 17D illustrates average pressure readings (e.g., 6 second averages), and also indicates target pressures, at a plurality of locations (upper, middle, lower). This way a user can know if they might want to inflate one or more bladders more to get closer to the target pressure for that particular location. Each individual bladder can be independent controlled in some embodiments. FIG. 17E illustrates a compliance screen, which is a compliance calendar with dates and percent compliance relative to target hours. In FIGS. 17C-E the alarm on/off icon can also be seen.

FIGS. 17C-E also illustrate additional functionality of this exemplary app. There is a real-time sensing mechanism to sense if the brace is being worn, and the visual display can indicate if it's of or off, based on the sensing. The visual display also includes how many hours the brace has been worn, and can be based on a given day. The visual display also presents the target time for wearing the brace (e.g., in hours). The visual display also displays the battery level remaining in the brace, and whether the alarm setting is on or off.

The alarm can turn on if pressure and/or force drops below a threshold value, and can advise the wearer to inflate fluid and/or tighten the brace.

In some embodiments the chassis (e.g., element 16 in FIG. 14) includes a pressure sensor that is adapted to sense pressure in the 0-5 PSI range. In some embodiments the force sensor is adapted to sense between 0-25 pounds. In some embodiments the chassis includes a 3-axis accelerometer, which can be used to determine patient position. The chassis can also include a battery to power the on-board components. The chassis can include any suitable microcontroller, and any suitable data storage device.

In any embodiment herein the fluid bladder can be PVC or any other suitable material. In any embodiment herein the bladder can have a diameter from 1 inch and 7 inches (e.g., from 2 to 6 inches, from 3-4 inches). The bladders can have other shapes, such as trapezoids. The bladders have any number of ports (e.g., 1, 2, 3). The bladder can be in communication with any type of inflation mechanism, such as a hand pump, or finger pump and release.

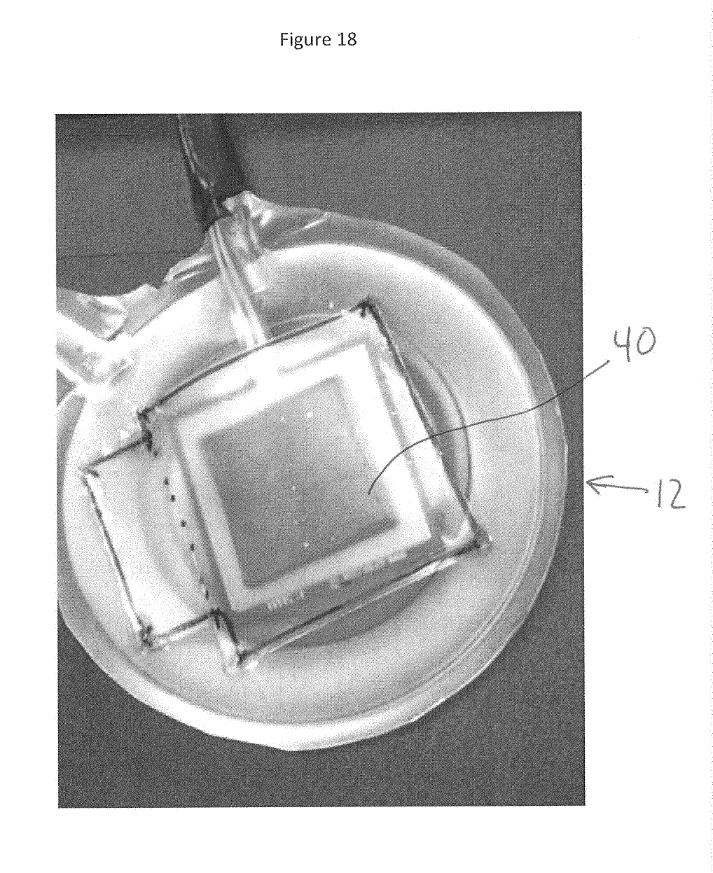

The force sensor/bladder assembly can include an integrated pocket sized and configured to receive therein the force sensor. FIG. 18 illustrates an integrated pocket 40 with a force sensor therein. In any of the embodiments herein the force sensor can be a force sensitive resistor, but other types of force sensors can be used.

In any of the embodiments herein the pressure sensor can be incorporated into the bladder.

The chassis in any of the embodiments herein can have any suitable configuration, such as rectangular or half-oval. It can be manufactured using any suitable technique, such as 3D printed or over molded. A chassis with at least one curved surface can reduce the footprint, reduce sharp edges, and provide flexibility for highly curved brace mounting.

The disclosure below builds on the disclosure above and is related thereto. Any aspect of the disclosure below may be incorporated into any suitable aspect of the disclosure above, including devices, systems, and/or methods.

With any of the restoring pads herein (which may also be referred to herein as pads, or the force/bladder assemblies, etc.), the patient is provided with a device that seamlessly fits within their brace to improve comfort and provide focal correction while adding proven support tools to improve compliance. In any of the embodiments herein, the pads can be a foam-covered inflatable pad adapted to sense force and pressure that can be wirelessly communicated (e.g., BlueTooth) to a patient's external device (e.g., smartphone), examples of which are described herein. In any of the embodiments herein, a finger pump on the inside surface of the brace can be used to control inflation and deflation of the bladder. The disclosure includes working prototypes and a working iOS app to engage and notify the patient if they are within their goals for pressure, force and wear-time, examples of which are described above. The pads improve comfort and increased the correcting forces in the brace.

At least two generations and sizes of bladders have been prototyped. The foam covering can be standard foam from orthotists' work-flow—same material, same adhesives, and installed at the same location they presently place pads—same size, same location.

The embodiments can be modified to include any of the following: device footprint reduction, soldered prototype converted to a custom fab board, battery optimization, Android app development and testing to US (IEC 60601) and International Standards.

Any of the pads herein can be used in any new or existing therapeutic braces (e.g., Rigo-Cheneau, Boston Brace). The pads herein are brand agnostic. Existing orthotists presently place foam/pads in the same location that the pads herein can be placed—e.g., at the apex of the patient's curve.

The forces can be managed momentarily (real-time), daily, or, for example, over the 6 months in between clinical visits.

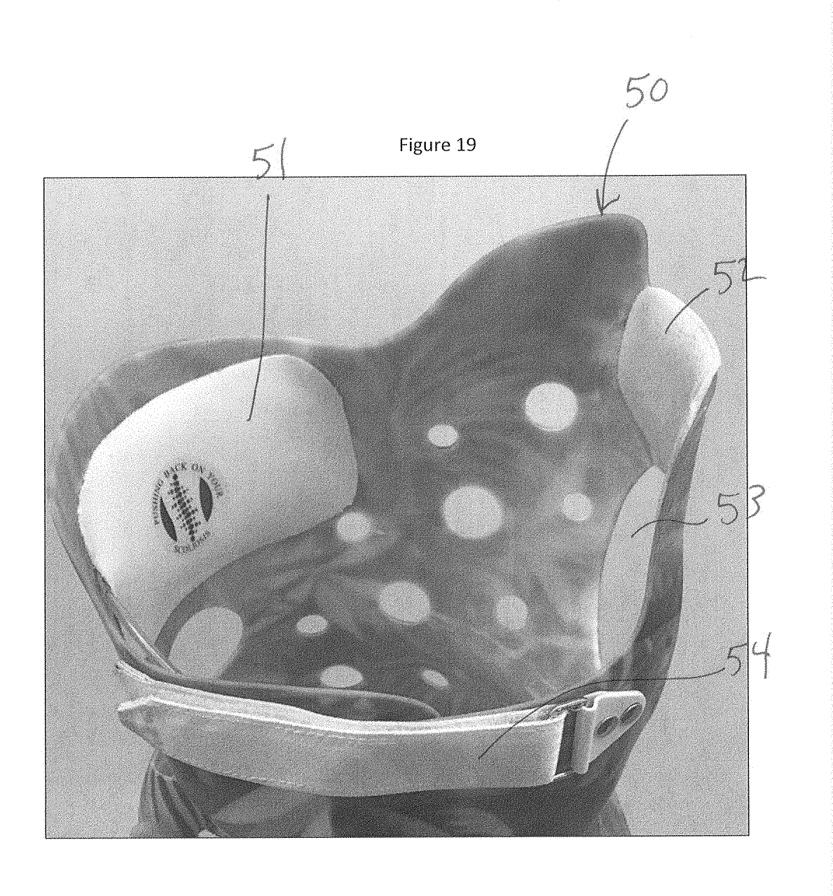

Exemplary pads 51, 52, and 53 and locations are shown in FIG. 19, in which the pads are secured to the inner surface of brace 50. The pads can include any of the components described herein—e.g., bladder, force sensor, communication component, etc. Any of the pads herein can be adapted to wirelessly communicate force and/or pressure information to an external device Eliminating wires can significantly ease the process during diagnostic or therapeutic procedures.

An advantage of the pads herein is that the conforming feature allows the patient to increase and decrease the volume of the pad. This is critical as patient specific geometry varies daily by body position and may change over weeks after a trauma or as inflammation subsides. For scoliosis patients in particular the geometry change may be over years as the most common patient diagnosed with adolescent idiopathic scoliosis is between 9-14 years and is actively associated with growing spurts. In this case the location within their orthotic and their body is a moving dynamic target. The ability to have the patient adjust their comfort is of importance to maintain fit.

Any of the sensors herein can be, but not limited to, force sensitive resistors, pneumatic sensors, load cells, and thermal sensors.

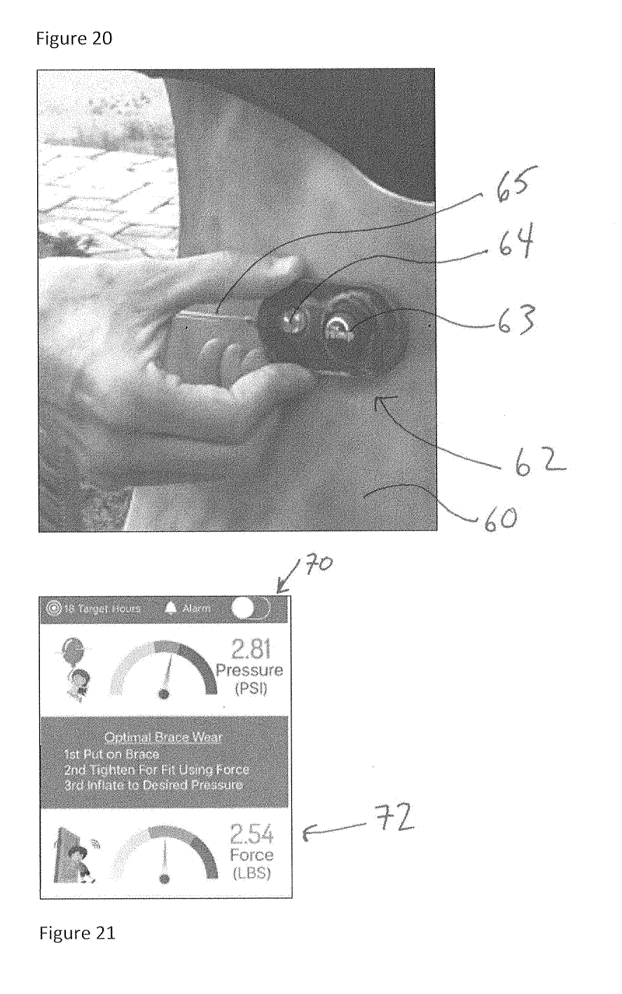

FIG. 20 illustrates an exemplary finger pump that can be used with any pad herein. Pump 62 includes pump button 63, fluid release button 64, and fluid line 65. Brace 60 can also be seen, to which the pump 62 is secured.

FIG. 21 illustrates an exemplary app screen that can show pressure and force readings, and also qualitatively on a spectrum for ease of use.

The software herein can log the data, and our web-enabled app communicates with the electronics on the orthotic/brace/cast and provides the patient a way to quantify their preferred wearing pressure and forces and seamlessly provides their performance. The patient is growing and the fit may become uncomfortable. If they are waiting 6 months until their next visit they won't get the benefit and their scoliosis may worsen. With the software herein we have the data to intervene and offer to find out what is changing in their care. It may be a simple reminder or a more importantly a window to advise moving up a fitting or new brace due to growth. This is not an option to intervene today like this one.

In some embodiment the restoring pads may also be interconnected to provide consistent pressure across multiple pads. They may be independent to optimize for fit. They may be adhered to the brace/orthotic with normal adhesives or rivets and other common means for brace fixation/attachment. They may also be provided with a way after 1 month to inflate a second bladder to increase the thickness or area of support as the patient continues to support/correct their own brace. In any of the embodiments herein they may alternatively be embedded within the surfaces of the existing brace material. As a pad normally sits on the top of the brace, we may integrate to the geometry conforming bladder is within the material of the brace as well.

Any of the restoring pads herein can also be used or integrated into any of the diagnostic systems, devices, and methods of use described herein.

The disclosure herein also provides for systems for and methods of identifying the location of and forces sufficient to reduce pain, which can be diagnostic in nature. The diagnostic devices herein, such as in the embodiment in FIG. 1, can also be or alternatively used to identify the location(s) and/or forces sufficient to reduce pain. For example, a subject with back/spine pain can stand within the system, and the distender interfaces can be modified to contact the subject. At least one of the interfaces can include a bladder and sensor as described herein. The at least one bladder can then be inflated while sensing force and/or pressure. Force and/or pressure can be monitored as set forth herein, and pain level can be assessed during the procedure (e.g., continuously). Pain level assessment can include the subject indicating pain level on almost any type of scale, such as 1-10; pain/no pain (binary), etc. When an acceptable amount of pain relief is achieved, the force and/or pressure can be recorded (using any device, component, or method herein), and this allows a brace to be more efficiently made (as set forth herein) that can reduce the subject's pain when the brace is worn. Thus, any of the diagnostic approaches herein can additionally (or alternatively) be used as part of a diagnostic assessment that can be used to create a therapeutic brace that can be used to treat pain (which can also be used to treat scoliosis as well).

The disclosure herein also provides methods of and systems adapted to monitor compliance and provide compliance information to a subject. The compliance of a wearable device, such as a therapeutic scoliosis brace, can be determined with the use of a thermal sensor secured to the wearable device, wherein the thermal sensor is set to close to body temperature threshold. So when a subject is wearing the wearable device, the thermal sensor senses the subject's body temperature, and can provide input to a computer executable method that can determine how long the subject was wearing the device over a given period of time (e.g., a day).

An alternative method of calculating compliance of a wearable device can be to utilize one or more biological dynamic variables, such as the subject's respiration cycle and ballistic cardiography. For example, when a subject is wearing any of the therapeutic braces herein, the force and/or pressure sensors will generally be able to sense a cyclical increase and decrease in the force and/or pressure as the subject exhales and inhales. Inhalation will cause an increase in sensed force and/or pressure, while exhalation will cause a drop in sensed force and/or pressure. A computer executable method can be adapted to analyze the sensed force and/or pressure over time, and detect the cyclical increase and decrease, providing an indication that the wearable device is being worn. An aggregate period of time during which the wearable device is worn can be determined, and the method can output to a display on a user's device information that is indicative of compliance. Other biological dynamic variables may also be able to provide an indication of compliance.

Ballistic cardiography has also confirmed that patient compliance can be sensed with an accelerometer alone, wherein the accelerometer can determine body position, and that the wearable device is being worn about the body. For example, a wearable device can have an accelerometer secured thereto. The accelerometer can provide input to a computer executable method that can determine that the wearable device is being worn about a subject. The same compliance calculations set forth above can be used, and the compliance can be output to a display on the user device.

It is also of note that patient outcomes and feedback have been advanced in the field of adolescent diabetes. Randomized Control Trials in self-management for improved outcomes has validated text messages support to be the most beneficial nudge in a recent study. “Text Message Intervention for Teens with Type 1 Diabetes (T1D) Preserves A1C: Results of an RCT,” presented Jun. 10, 2017, at the American Diabetes Association's 77th Scientific Sessions® at the San Diego Convention Center. The investigators reported “The research indicated that text message reminders appeared to preserve and potentially improve glycemic control. Additionally, in a multivariate mixed linear model, the frequency of response to text messages significantly predicted A1C levels (p<0.003).”

What is claimed is:

1. A force and/or pressure sensing pad adapted for use in spinal diagnostic and therapeutic methods, comprising:
an inflatable bladder adapted to be in communication with a fluid source for at least one of inflation and deflation;
a force sensor co-located with the inflatable bladder and adapted to sense force, the force sensor in communication with a communication component, wherein the communication component is adapted to at least one of store data and upon availability wirelessly communicate information indicative of the sensed force to a remote device.

2. The force sensing pad of claim 1, further comprising an adhesive substance and a removable cover on at least one portion of the pad, the removable cover adapted to be removed from the pad to expose the adhesive substance so that the pad can be adhered to a surface and at least temporarily adhered thereto with the adhesive substance.

3. The force sensing pad of claim 2, wherein the adhesive substance is adapted such that the pad can be removed from the surface if needed, such as for replacing with a second pad.

4. A force and/or pressure sensing pad adapted for use in spinal diagnostic and therapeutic methods, comprising
an inflatable bladder adapted to be in communication with a fluid source for at least one of inflation and deflation;
a force sensor co-located with the inflatable bladder and adapted to sense force;
an adhesive material; and
a removable cover, the removable cover positioned relative to the adhesive material such that removal of the removable cover exposes the adhesive material to the ambient environment such that the force sensing pad can be at least temporarily adhered to surface of another object.

5. The force sensing pad of claim 4, wherein the force sensor is in communication with a communication component, wherein the communication component is adapted to wirelessly communicate information indicative of the sensed force to a remote device.

6. A method of adhering a removable force sensing pad to a spinal diagnostic or therapeutic device, comprising:
providing a force sensing pad that includes an inflatable bladder co-located with a force sensor, and a removable cover;
removing the removable cover from the force sensing pad to reveal an adhesive substance on the force sensing pad;
positioning the adhesive substance against a diagnostic or therapeutic device to at least temporarily adhere the force sensing pad to a surface of the diagnostic or therapeutic device.

7. The method of claim 6, wherein the positioning step comprises positioning the adhesive substance against a therapeutic brace to treat scoliosis to at least temporarily adhere the force sensing pad to a surface of the therapeutic brace to treat scoliosis.

8. A method of providing information about force and pressure associated with a therapeutic scoliosis brace to a patient, comprising:
a computer executable method stored in a storage device of a remote device, the computer executable method adapted to:
receive information indicative of force sensed by a force sensor co-located with an inflatable bladder, the force sensor and inflation bladder coupled to a therapeutic scoliosis brace,
receive information indicative of fluid pressure in the inflatable bladder;
display on a display of the remote device information indicative of the sensed force as well as information indicative of the fluid pressure.

9. The method of claim 8, wherein the information indicative of the sensed force and information indicative of the fluid pressure comprises at least one of a quantity and a visual representation of the information.

10. The method of claim 8, wherein the information indicative of the sensed force and information indicative of the fluid pressure is visually presented relative to one or more thresholds.

11. A diagnostic apparatus for applying corrective forces on a patient's spine while sensing patient information, comprising:
a patient apparatus comprising a plurality of distenders each extending in a traverse plane an each supported by an upright support member, each of the distenders comprising a patient interface portion, each of the patient interface portions comprising a relatively rigid element that has a curvilinear configuration.

12. The apparatus of claim 11, wherein the relatively rigid elements that have the curvilinear configurations are sections of a traditional therapeutic scoliosis brace.

13. The apparatus of claim 11, further comprising a force sensing pad comprising an inflatable bladder and a force sensor, the inflatable bladder and force sensor secured directly or indirectly to an inner surface of the rigid element, and either permanently secured or temporarily secured thereto.

14. The apparatus of claim 11, wherein each of the relatively rigid elements has at least one degree of freedom (and optionally up to six degrees of freedom) relative to an elongate body of each of the distenders.

15. A diagnostic method of distorting a patient's spine as part of a brace making procedure, comprising:
engaging a torso of a patient (even if covered with clothing) with more than one distender interface, with a first distender interface engaging one side of the patient and a second distender interface engaging a second side of the patient, the first and second distender interfaces comprising, respectively, first and second relatively rigid elements that have a curvilinear configuration, and, wherein at least one of the first and second distender interfaces further comprises a bladder and a force sensor;
sensing pressure in the at least one bladder;
inflating the at least one bladder;
applying corrective forces on the patient with the at least one bladder; and
measuring forces from the at least one force sensor when the sensed pressure in the at least one bladder is below an upper pressure threshold.

16. A diagnostic method of distorting a patient's spine as part of an assessment to identify the location and forces sufficient to reduce pain, comprising:
engaging a torso of a patient (even if covered with clothing) with more than one distender interface, with a first distender interface engaging one side of the patient and a second distender interface engaging a second side of the patient, the first and second distender interfaces comprising, respectively, first and second relatively rigid elements that have a curvilinear configuration, and, wherein at least one of the first and second distender interfaces further comprises a bladder and a force sensor;
sensing pressure in the at least one bladder;
inflating the at least one bladder;
applying corrective forces on the patient with the at least one bladder; and
measuring forces from the at least one force sensor when the sensed pressure in the at least one bladder is below an upper pressure threshold.

17. A therapeutic method of distorting a patient's spine as part of an assessment of procedure to identify the location and forces sufficient to reduce pain, comprising:
engaging a torso of a patient (even if covered with clothing) with more than one distender interface, with a first distender interface engaging one side of the patient and a second distender interface engaging a second side of the patient, the first and second distender interfaces comprising, respectively, first and second relatively rigid elements that have a curvilinear configuration, and, wherein at least one of the first and second distender interfaces further comprises a bladder and a force sensor;
sensing pressure in the at least one bladder;
inflating the at least one bladder;
applying corrective forces on the patient with the at least one bladder; and
measuring forces from the at least one force sensor when the sensed pressure in the at least one bladder is below an upper pressure threshold.

18. A computer executable method of calculation of compliance of a wearable device, the method comprising:
sensing over a period of time at least one of force and pressure associated with an inflatable bladder secured to a wearable device configured to be worn about a subject's torso (optionally a therapeutic scoliosis brace);
determining that a detected cyclical increase and decrease in the at least one of sensed force and sensed pressure is due to a respiration cycle;
determining an aggregate amount of time during which the detected increase and decrease occurs;
calculating the subject's compliance time of wearing the wearable device within a given period of time, such as a day; and
optionally outputting information for display on a user device that is indicative of the subject's compliance.
